(19) United States
(12) Patent Application Publication
(10) Pub. No.: US 2023/0162857 A1
(43) Pub. Date: May 25, 2023

(54) BIOLOGICAL SIGNAL ANALYSIS ALGORITHM, SYSTEM, AND METHOD

(71) Applicant: Dong Whan LEE, Seoul (KR)

(72) Inventor: Dong Whan LEE, Seoul (KR)

(21) Appl. No.: 17/912,569

(22) PCT Filed: Mar. 15, 2021
(86) PCT No.: PCT/KR2021/003149

§ 371 Date: Sep. 19, 2022

(30) Foreign Application Priority Data
Mar. 31, 2020 (KR) 10-2020-0039248
Jul. 14, 2020 (KR) 10-2020-0086623

(51) Int. Cl. G16H 50/20; A61B 5/01; A61B 5/00

(52) U.S. Cl. CPC G16H 50/20; A61B 5/01; A61B 5/7264; A61B 5/0008; A61B 5/7282; A61B 5/4842; A61B 5/6844; A61B 5/002; A61B 5/0022; A61B 5/14542

(57) ABSTRACT

The present invention relates to a big data platform based analysis algorithm, system, and method for identifying, tracking, and preventing a target individual in a virus incubation period by using a terminal that collects biosignal measurement values and provides an epidemiological investigation according to digital anti-epidemic through a storage, distribution, collection, and analysis processing of the biosignal measurement data values.

### FIELD OF THE INVENTION

The present invention relates to an analysis algorithm, a big data platform based analysis algorithm, a system, and a method for identification, tracking, and prevention of a target individual in a virus incubation period, by minimizing errors and distortions of biosignal data values according to types of terminal that collect biosignals, user conditions, surrounding environmental conditions, collection methods, personal activities, and others; calculating an average value of measured body temperature values in a normal state that are collected after wearing a biosignal measurement terminal; classifying and reading viral infection stages by using a deviation value from the collected body temperature values; and analyzing prediction of a virus type, an estimated time of reaching an infection stage, and an estimated time of an onset of fever by using an increase and decrease rate of the deviation value over time.

### BACKGROUND OF THE INVENTION

Although the development of life science continues, economic losses and human casualties are increasing day by day due to the pandemic caused by the novel virus, COVID-19. As the development of preventive vaccines and new drug treatments for novel viruses is delayed, there is urgent needs for the introduction of technical methods, various epidemiological investigation methods, and quick response measures to emergency situations.

In addition, in order to prevent the spread of COVID-19 infection, monitoring body temperature is emerging as a new social issue. Although smart bands and smart watches that can continuously measure body temperature are being released, errors and distortions in measured body temperature values occur due to shapes of the terminals, user conditions, surrounding environmental conditions, collection methods, personal activities, and the like. In addition, there are serious problems in that there are no analysis algorithm that can identify an individual suspected of infection in the virus incubation period by tracking continuous body temperature measurement value and that there are a lot of asymptomatic infection in which fever is not recognized after viral infection.

Korean Patent Publication No. 10-1818857 [smart band type thermometer capable of measuring and monitoring body temperature] relates to a smart band thermometer capable of measuring and monitoring body temperature. This patent is provided with a band portion formed in a band shape of a certain width so as to surround the user's arm and is operated to provide a warning when a measured body temperature is higher or lower than a reference temperature, by comparing the body temperature measured from a temperature sensor and the reference temperature in a normal state set based on an average value derived from accumulated body temperature data. That is, this patent relates to a smart band-type thermometer capable of measuring and monitoring body temperature that provides continuous body temperature measurement.

However, when calculating a body temperature value in a normal state, the prior art simply calculates the average value by using the accumulated body temperature data and then compares and determines whether a body temperature is higher or lower than this average value. This method has a disadvantage in that errors and distortions in body temperature may occur depending on the shapes of the terminals, environmental conditions, collection methods, personal activities, and the like. In addition, the prior art does not disclose a temperature acclimatization process between the sensing element and the skin surface when the smart band is first worn and a specific technical method of calculating body temperature measurements in a normal state.

Accordingly, the present invention is clearly distinguished from the prior art in that it performs an acclimatization process for temperature acclimatization between a sensing element and a skin surface for a certain period of time when wearing a smart band; measures body temperature several times at intervals for a certain time within an error range of ±0.5° C.; and calculates an average value of body temperature in a normal state, excluding the highest and lowest temperature of the collected measured temperature values, to read an infection stage by using a deviation value from the collected body temperature value, to predict a virus type by using a rate of increase or decrease of the deviation value over time, to track an estimated time of reaching an infection stage or an estimated time of an onset of fever, and to notify data information including location information to an application of a mobile terminal when an event that an individual suspected of being infected with a virus occurs.

### DETAILED DESCRIPTION OF THE INVENTION

### Technical Problems

The present invention has been made to solve the problems of the prior art, and an object of the present invention is provided to an analysis system and an algorithm for minimizing errors and distortions in body temperature measurement values collected from a terminal, classifying and reading viral infection stages, and identifying, tracking, and preventing a target individual in the virus incubation period based on a big data platform.

In addition, other technical problem and object to be solved by the present invention are to provide a technical method that can remotely monitor an individual suspected of being infected with novel viruses such as COVID-19 by identifying and locating them and that can conduct various epidemiological investigations through a digital anti-epidemic system based on a big data platform.

Technical problems and objects to be solved by the present invention are not limited to the technical problems and objects mentioned above, and other technical problems and objects not mentioned will be clearly understood by those skilled in the art from the following description.

### SUMMARY OF THE INVENTION

In order to achieve the object mentioned above, the present invention further comprises a server of receiving a measured body temperature value that is analyzed from a mobile terminal to determine in an application of the mobile terminal whether an individual is a suspected target, by analyzing a measured body temperature value transmitted from a Bluetooth module of a biosignal measurement terminal, while minimizing errors and distortions of collected body temperature values according to a shape of the terminal, a user condition, a collection method, and a surrounding environmental condition.

After downloading a mobile terminal application, the present invention can register information and set an access distance to a target individual suspected of infection who wears the terminal to prevent viral infection; can calculate an average body temperature in a normal state that is measured several times at regular time intervals in a static state; and can read a viral infection stage by using a deviation value from the measured body temperature value.

In addition, the present invention can track and stop tracking changes in body temperature by using an increase and decrease rate of the body temperature deviation value over time and can calculate an expected time of reaching body temperature in an infection stage and an expected time of an onset of fever at each infection stage, by calculating a significant increase and decrease rate through tracking and stopping tracking a change in body temperature.

In addition, when an event of an individual suspected of viral infection occurs in the mobile terminal application, the present invention can notify to the server an information value including multiple access location information of the mobile terminal application of a terminal worn on an individual as a form of text, image, voice, and others. Furthermore, the present invention can identify, track, isolate, and prevent an individual suspected of infection in the virus incubation period based on the big data platform.

The features and advantages of the present invention will become more apparent from the following detailed description in conjunction with the accompanying drawings.

### Technical Effects of the Invention

The present invention can identify, track, and prevent a target individual in a virus incubation period through a biosignal measurement terminal and can build big data through deep learning and machine learning based on a big data platform by utilizing a biosignal analysis algorithm when an event occurs in a target individual wearing a terminal.

In addition, the present invention can calculate an expected time of reaching body temperature in an infection stage and an estimated time of an onset of fever in the infection stage, by using an increase and decrease rate of a body temperature deviation value over time. Furthermore, the present invention can track a change in body temperature or stop tracking and can calculate a significant increase and decrease rate through tracking and stopping tracking of a change in body temperature. In addition, the present invention can transmit information data value of an individual suspected of viral infection in the mobile terminal application to the server when an event is occurred in the terminal worn on the individual and can prevent viral infection by notifying information value including multiple access location information of the mobile terminal application as a form of number, text, image, voice, and others to the mobile terminal from the server.

In addition, the present invention can identify and track an individual suspected of viral infection by tracking a minute change in body temperature in the incubation period of an asymptomatic infected individual.

In addition, the present invention can transmit to and receive from the server various biosignal measurement data values collected from the terminal and can prevent viral infection through intact medical supports that can receive treatment, diagnosis, and prescription by utilizing the measured big data of each biosignal received and stored in the server.

### BRIEF DESCRIPTION OF THE DRAWINGS

FIGS. 1a and 1b are graphs schematically illustrating a correlation between the number of virus population and oxygen saturation.

FIG. 1c is a graph schematically illustrating a change in a deviation value according to an increase in body temperature after virus inoculation.

FIG. 2 is a block diagram schematically illustrating a system configuration of the present invention.

FIG. 3 is a terminal in a worn state and a perspective view illustrating a shape of a terminal of the present invention.

FIG. 4 is a conceptual diagram of a multiple access location tracking system of the present invention.

FIG. 5 is a flowchart schematically illustrating a processing state of a biosignal measurement value of the present invention.

FIG. 6 is a flowchart illustrating a transmission state of a biosignal measurement data in a mobile terminal of the present invention.

FIG. 7 is a flowchart schematically illustrating a process of classifying viral infection stages of the present invention.

FIG. 8 is a block diagram illustrating reading of an infection time point of an individual suspected of viral infection of the present invention.

FIG. 9 is a flowchart illustrating a data flow state between a biosignal measurement terminal and a server according to the present invention.

FIG. 10 is a graph schematically illustrating a method of calculating a deviation value and an increase and decrease rate of body temperature according to the present invention.

FIG. 11 is a graph schematically illustrating tracking of an estimated time of reaching body temperature in an infection stage and an estimated time of a starting point of fever in the infection stage of the present invention.

FIG. 12a is a graph schematically illustrating a method of tracking a change in body temperature according to the present invention.

FIGS. 12b to 12e are graphs schematically illustrating a method of stopping tracking of body temperature changes according to the present invention.

FIG. 13 is a graph schematically illustrating a method of determining whether measured body temperature is normal according to the present invention.

FIG. 14 is a graph schematically illustrating a method of predicting a virus type of the present invention.

### MODES FOR THE INVENTION

Hereinafter, preferred embodiments of the present invention will be described with reference to the accompanying drawings. In this process, the thickness of lines or the size of components shown in the drawings may be exaggerated for clarity and convenience of explanation.

In addition, terms to be described below are terms defined in consideration of functions in the present invention, which may vary according to intentions or customs of users and operators. Therefore, definitions of these terms should be made based on the description throughout this specification.

The accompanying drawings are illustrated by exaggerating or simplifying for convenience and clarity of explanation and understanding of configuration and operation of the technology, and each component does not exactly match the actual size and shape.

The embodiments described below are provided to fully inform those of ordinary skill in the scope of the invention, and the present invention is not limited to the embodiments disclosed below and may be embodied in various forms.

Like elements in the drawings refer to like reference numbers. Specific details in the following description are provided to help a more general understanding of the present invention and are not intended to limit the present invention to specific embodiments. That is, all changes included in the spirit and scope of the present invention should be understood to be include in equivalents or substitutes of the present invention. In the description of the present invention, if it is determined that a detailed description of a related known function or configuration may unnecessarily obscure the gist of the present invention, the detailed description thereof will be omitted.

In addition, the following examples do not limit the scope of the present invention but are merely exemplary embodiments of components presented in the claims of the present invention. That is, embodiments including an element that is included in the technical spirit of the present invention and that is substitutable as equivalents in the elements of the claims may be included in the scope of the present invention.

In more detail, a system of the present invention comprises: a mobile terminal application analyzing a measured body temperature value transmitted from a Bluetooth module of a biosignal measurement terminal to determine whether an event has occurred; a server receiving the measured body temperature value analyzed in the mobile terminal application when the event occurs; and at least one of a mobile terminal receiving and wirelessly transmitting location data and body temperature measured from the biosignal measurement terminal and a gateway receiving and transmitting the location data and the body temperature measured from the biosignal measurement terminal. The server receives and stores the location data and the body temperature value measured from the biosignal measurement terminal from at least one of the mobile terminal and the gateway; receives the location data and the body temperature value measured from the biosignal measurement terminal when the event occurs; includes multiple access location information of the mobile terminal application; and notifies a number, text, image, and voice of individual information of the event. The server further includes: a database unit analyzing and storing the body temperature value measured from the biosignal measurement terminal and a transceiver unit capable of transmitting the measured body temperature value through an internet network. That system can build big data through machine learning and deep learning of body temperature measurement values generated by the event.

In addition, the present invention provides an analysis method for identifying, tracking, and preventing a target individual in the virus incubation period based on a big data platform. Specifically, an algorithm of analyzing the body temperature value measured from the biosignal measurement terminal comprises the steps of: calculating a body temperature value in a normal state by using an analysis algorithm of the body temperature value measured from the biosignal measurement terminal; determining whether the measured body temperature value is normal; classifying an infection stage by using a deviation value between the calculated body temperature value in the normal state and a subsequently measured body temperature value; calculating a deviation value and an increase and decrease rate of the measured body temperature value; tracking an expected time of reaching to body temperature in an infection stage and an expected time of an onset of fever in the infection stage; tracking a change in body temperature by using the increase and decrease rate of the deviation value of body temperature; stopping the tracking of the change in body temperature; and predicting a virus type through an increase and decrease of the measured body temperature value.

Hereinafter, preferred embodiments of the present invention will be described in more detail with reference to the drawings.

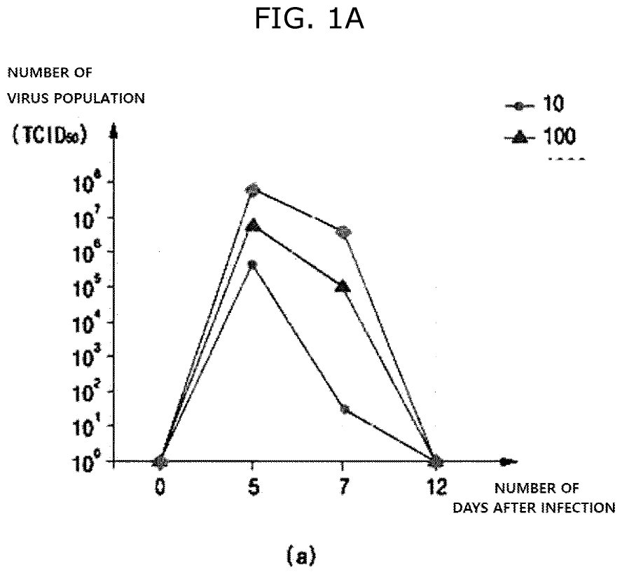

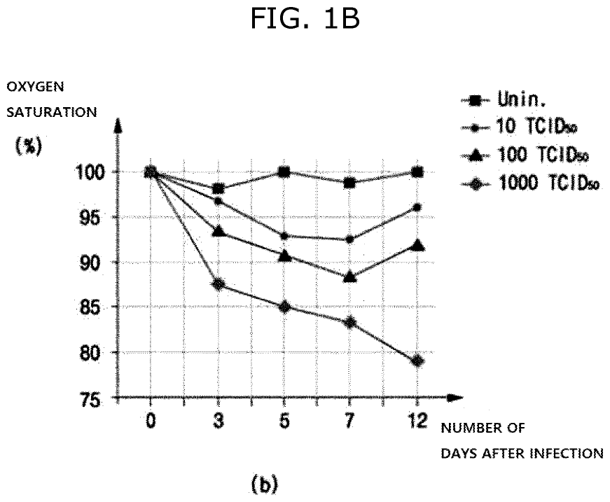

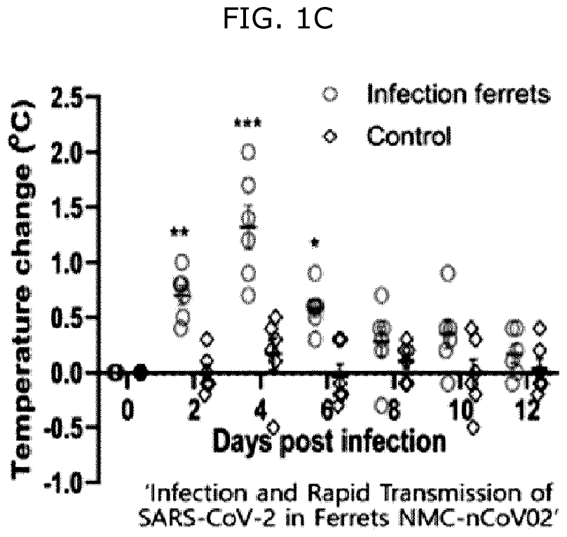

FIG. 1a is a graph schematically illustrating a correlation between the number of a virus population and oxygen saturation. Referring to FIG. 1a, in an experiment on ‘Pulse-oximetry accurately predicts lung pathology and the immune response during influenza infection’ published in the US National Library of Medicine National Institutes of Health (PMC2776688), viral infection induces local immune and inflammatory responses leading to epithelial damage and pneumonia (Taubenberger, 2008). In addition, as shown in FIG. 1B, an experiment evaluated whether an oxygen saturation (SaO2) level is directly related to lung pathology at all stages of infection, and whether the oxygen saturation level can be a useful indicator of the severity of influenza infection depending on the number of virus populations. In the above experiment, Influenza A/PR8/34 (PR8) virus was intranasally infected into BALB/c mice with concentrations of 10TCID₅₀, 100TCID₅₀, and 1000TCID₅₀, respectively, and FIG. 1B illustrates a correlation between the number of virus population and oxygen saturation according to the number of days after infection.

Therefore, as shown in FIG. 1a, the mice infected with virus concentrations of the 10TCID₅₀, 100TCID₅₀, and 1000TCID₅₀ respectively showed a peak in the number of viruses on the fifth day after infection, and the number of viruses per ml were detected as 10⁵ to 10⁶, 10⁶ to 10⁷, and 10⁷ to 10⁸, respectively. As shown in FIG. 1B, the experiment showed a correlation that the oxygen saturation after infection with the respective virus concentrations of 10TCID₅₀, 100TCID₅₀, and 1000TCID₅₀ gradually decreased with the number of days elapsed. In addition to the mouse experiments, in animal experiments with monkeys, weasels, pigs, dogs, and cats, there was a correlation in which oxygen saturation decreased as the number of virus population increased.

FIG. 1c is a graph schematically illustrating a change in a deviation value according to an increase in body temperature after a challenge inoculation of the SARS-CoV-2 (COVID-19) strain in an animal experiment with weasels. Referring to FIG. 1c, in an experiment related to COVID-19 of a paper ‘Infection and Rapid Transmission of SARS-CoV-2 in Ferrets NMC-nCoV02’ published on Mar. 23, 2020, at a website, “https://doi.org/10.1016/j.chom,” in order to confirm transmission between weasels, weasels (n=2) were intranasally inoculated (IN) with a virus of 10^{5.5}TCID₅₀ of the strain (NMC-nCoV02) isolated from a patient diagnosed with COVID-19 in Korea in February 2020. As an experimental method, direct contact (DC) by breeding uninfected and infected weasels in one place or indirect contact (IC) by isolating the uninfected weasel from the infected weasel and using a permeable partition between them were performed, and the number of individuals infected with SARS-CoV-2 on the second day after the first challenge inoculation was recorded. This study was repeated in 3 independent experiments (total n=24; direct infection [n=6], DC[n=6], IC[n=6], and PBS control [n=6]). The body temperature of the weasel infected with NMC-nCoV02 increased from 38.1° C. to 40.3° C. for about 2 to 8 days after the challenge inoculation, and the body temperature increased in all weasels infected with 6 direct infections (DC). There was no change in body temperature increase until a latent period for a certain period of time after viral infection, but the onset of fever started after about 36 hours.

Therefore, the experimental result showed that body temperature increased according to the number of days elapsed from an onset of fever after reaching a quantitative threshold for the number of virus population in the blood as the virus passes through the latent period according to an increase in the number of viruses after viral infection.

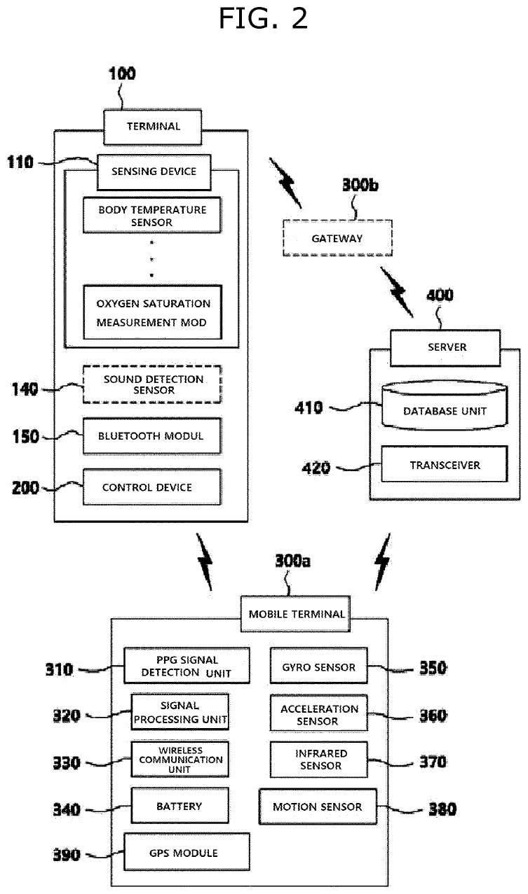

FIG. 2 is a block diagram schematically illustrating a system configuration of the present invention. Referring to FIG. 2, a wearable type biosignal measurement terminal 100 of the present invention includes a control device 200 provided on one surface of a body of the biosignal measurement terminal 100; one or more sensing elements 110 including an infrared sensor, a body temperature sensor, and an LED, and capable of implementing sensing technologies such as optical blood flow measurement and pulse oximetry; and a Bluetooth module 150 connected to a mobile terminal or a tablet. The control device 200 is provided integrally with the terminal 100 or is provided to be detachably attached to the terminal 100.

In addition, the system of the present invention is further provided with at least one of a mobile terminal 300a receiving and transmitting location data and biosignal measurement data value wirelessly; and a gateway 300b receiving and transmitting the location data value and the biosignal measurement data value from the terminal 100. The system includes a server 400 that receives and stores the location data value and the biosignal measurement data value from at least one of the mobile terminal 300a and the gateway 300b and that analyzes and reads the location data value and each biosignal measurement data received when an event occurs to generate a reading information value for an individual who wears the terminal. The server 400 further includes a database unit 410 storing the received biosignal sensing data value, and a transceiver 420 transmitting the biosignal sensing data value through the internet network and may build a platform based on artificial intelligence through machine learning and deep learning with big data of measured biosignal values related to the event.

In addition, the measured data value obtained from the sensing element 110 of the terminal 100 including the control device 200 is transmitted to the server 400 through at least one of the mobile terminal 300a and the gateway 300b.

According to an embodiment of the present invention, the mobile t31erminal 300a of the individual wearing the terminal 100 includes a battery 340, a gyro sensor 350, an acceleration sensor 360, an infrared sensor 370, and a motion detection sensor 380, a GPS module 390, and the like. The mobile terminal 300a can set a measurement time and the number of times of the sensing device 110 of the terminal 100 on a screen of an application of the terminal 100 by using the gyro sensor 350, the acceleration sensor 360, the motion sensor 380, the GPS module 390, and others. At this time, since the mobile terminal 300a collects the sensing data value obtained from the sensing element 110 only in a static state, more accurate biosignal sensing data values can be collected.

In addition, the mobile terminal 300a of the individual wearing the terminal 100 further includes: a PPG signal detection unit 310 detecting a PPG (Photo Plethysmo Graphic) signal when collecting the biosignal sensing data value; a signal processing unit 320 that enables measurement in a static state by using the acceleration sensor 360 and the gyro sensor 350 and that amplifies and digital converts the PPG signal and the static signal for detecting a static signal; and a wireless communication unit 330 that processes and transmits the digitally converted PPG signal and the static signal according to a wireless communication standard.

As an example of an application of the terminal 100 of the present invention, when a passenger riding on transportation, such as an airplane, a ship, a train, a bus, or a subway, wears the terminal 100, the passenger's biosignal measurement values, such as an increase in body temperature, an increase in respiration rate, and a decrease in oxygen saturation, are collected, and accordingly, it is possible to identify and track an individual suspected of viral infection. Virus infection can be prevented by wearing the terminal 100 in dense places, such as military bases, kindergartens, schools, companies, theaters, performance halls, churches, cathedrals, temples, factories, and gathering places.

In addition, the system and method of the present invention can identify and track an individual suspected of viral infection by transmitting data values using various measurement technologies through 3G, LTE, 5G communication, and others, processing the data in the server 400, storing the data in the database unit, and configuring the data as a DB system, and analyzing the results of the stored data. In addition, the terminal 100 may measure, collect, and analyze biosignal sensing data values, such as electromyography, respiration rate, electrocardiogram, blood pressure, pulse rate, and activity level, including oxygen saturation, body temperature, and frequency of cough sound. The above technology is commonly known to those in the field of the present invention, and a detailed description thereof will be omitted.

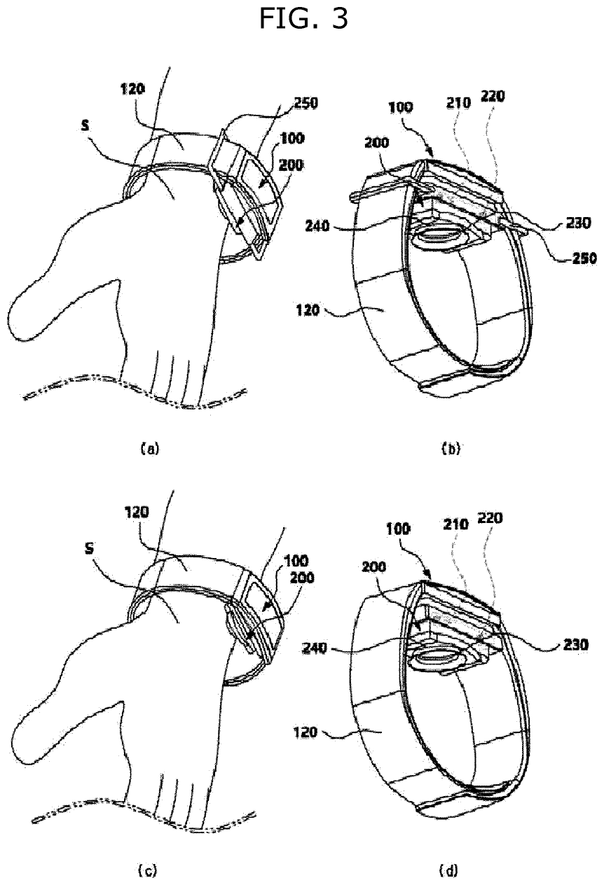

FIG. 3 is a terminal in a worn state and a perspective view illustrating a shape of the terminal 100 of the present invention. Referring to FIG. 3, for convenience of explanation, one surface of the terminal 100 in a direction in contact with the wearer's skin S refers to as a rear surface, and the other surface provided in a direction opposite to the rear surface is referred to as a front surface.

Referring to (a) of FIG. 3, the control device 200 is included in the terminal 100 in order to minimize errors and distortion of biosignal measurement data values. The control device 200 is attached to a main body of the terminal 100 by a connection member 250 and has a structure that is easy to attach and detach from the terminal 100.

Referring to (b) of FIG. 3, the control device 200 for minimizing errors and distortions of the biosignal measurement data value, includes: a storage space 210 formed on a rear side of a central part of the terminal 100 so as not to interfere with sensing of biosignals of the terminal 100; an elastic spring 220 mounted in the storage space 210 and maintaining a predetermined distance between various sensing elements and a skin contact surface; an open type chamber 230 provided at a lower end of the control device 200 and having the same curvature as the contact area of the skin surface S; a chamber mounting groove 240 concavely formed on a bottom surface to have a shape corresponding to that of the open type chamber 230; and a connection member 250 connecting the control device 200 and the terminal 100. The shape of the connection member 250 may perform a function of connecting the control device 200 and the terminal 100. In addition, referring to (c) and (d) of FIG. 3, the terminal 100 may include various shapes and structures to perform the functions of (a) and (b) of FIG. 3 without the connection member 250. For example, the terminal 100 may be integrally formed with the control device 200. Since integrated structure is a commonly known technology to those in the field of the present invention, a detailed description will be omitted.

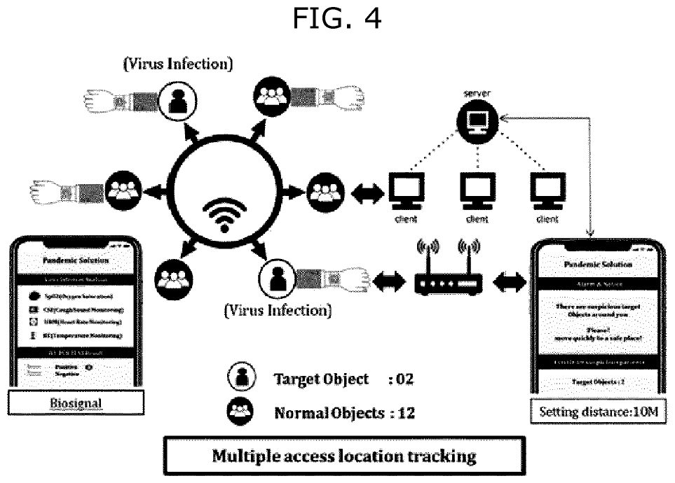

FIG. 4 illustrates a system of preventing viral infection by using multiple access location tracking. Referring to FIG. 4, after the mobile terminal 300a downloads an application, the server 400 sets an access distance (10 M) from the mobile terminal 300a to a target individual suspected of infection who wears the terminal 100 in order to prevent viral infection. The server 400 collects multiple access location information data values of the mobile terminal application, and when an event occurs in a crowded area, the server 400 transmits to the mobile terminal application the information values including the location information between the target individual suspected of infection wearing the terminal 100 and the mobile terminal 300a and thus can notify the number of individuals suspected of viral infection on a screen of the mobile terminal application. According to the described structure, it is possible to prevent viral infection by using location tracking through real-time GPS.

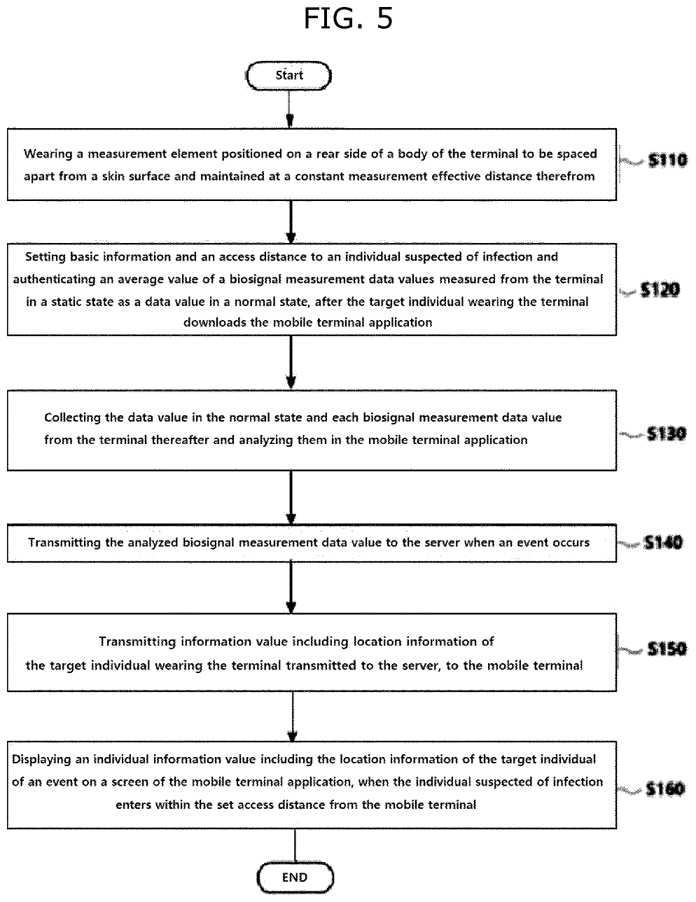

FIG. 5 is a flowchart schematically illustrating a processing operation of a biosignal measurement data value according to the present invention. Referring to FIG. 5, the processing operation of a biosignal measurement data value includes the steps of: wearing the measurement element positioned on a rear side of a body of the terminal 100 to be spaced apart from the skin surface and maintained at a constant measurement effective distance therefrom S110; setting basic information and the access distance to the individual suspected of infection and authenticating an average value of the biosignal measurement data values measured from the terminal 100 in a static state as a data value in a normal state, after the target individual wearing the terminal 100 downloads the mobile terminal application S120; collecting the data value in the normal state and each biosignal measurement data value from the terminal 100 thereafter and analyzing them in the mobile terminal application S130; transmitting the analyzed biosignal measurement data value to the server 400 when an event occurs S140; transmitting information value including the location information of the target individual wearing the terminal 100 transmitted to the server 400 to the mobile terminal 300a, S150; displaying an individual information value including the location information of a target individual of an event on a screen of the mobile terminal application, when the individual suspected of infection, who wears the terminal 100, enters within the set access distance from the mobile terminal 300a, S160. The server 400 may continuously track multiple access location of the application and may have a parallel distributed data processing structure capable of storing, distributing, collecting, and analyzing biosignal measurement data values of target individuals of events.

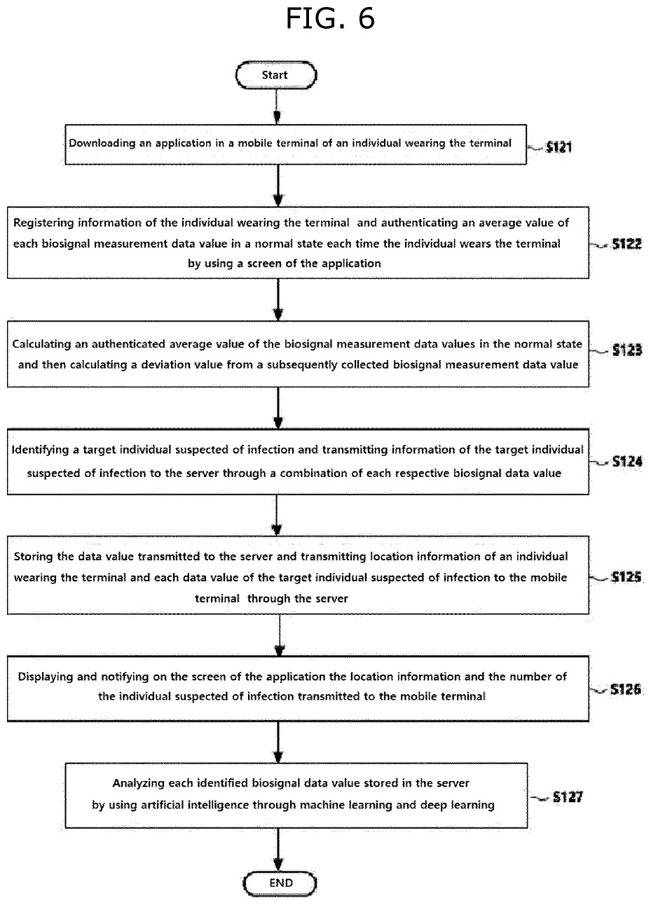

FIG. 6 is a flowchart illustrating an operation of transmitting biosignal measurement data in a mobile terminal 300a of the present invention. Referring to FIG. 6, the operation of transmitting biosignal measurement data in the mobile terminal 300a includes the steps of: downloading an application in the mobile terminal 300a of an individual wearing the terminal 100 S121; registering information of the individual wearing the terminal 100 and authenticating an average value of each biosignal measurement data value in a normal state each time the individual wears the terminal 100, by using a screen of the application S122; calculating an authenticated average value of the biosignal measurement data values in the normal state and then calculating a deviation value from a subsequently collected biosignal measurement data value S123; identifying a target individual suspected of infection and transmitting information of the target individual suspected of infection to the server 400 through a combination of each respective biosignal data value S124; storing the data value transmitted to the server 400 and transmitting location information of an individual wearing the terminal 100 and each data value of a target individual suspected of infection who wears the terminal 100 to the mobile terminal 300a through the server 400, S125; displaying and notifying on the screen of the application the location information and the number of the individual suspected of infection transmitted to the mobile terminal 300a, S126; and analyzing each identified biosignal data value stored in the server 400 by using artificial intelligence through machine learning and deep learning S127. According to the described structure, the server 400 can establish digital anti-epidemic system capable of remotely monitoring epidemiological investigations such as a source of infection, a route of infection, and a transmission rate of infection, by receiving the measurement values measured by the terminal 100 from the mobile terminal 300a and builds big data based on the measurement values.

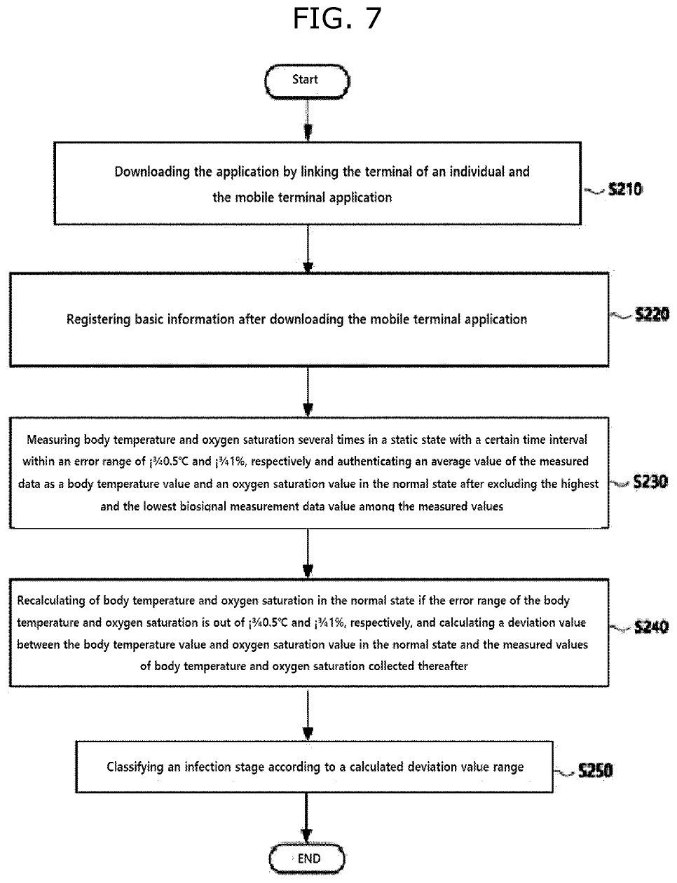

FIG. 7 is a flowchart schematically illustrating a process of classifying infection stages of the present invention. Referring to FIG. 7, the process of classifying infection stages includes the steps of: downloading the app by linking the terminal 100 of an individual and the mobile terminal application S210; registering basic information and others after downloading the mobile terminal application S220; measuring body temperature and oxygen saturation several times in a static state with a certain time interval within an error range of ±0.5° C. and ±1%, respectively and authenticating an average value of the measured data as a body temperature value in a normal state and an oxygen saturation value in the normal state after excluding the highest biosignal measurement data value and the lowest biosignal measurement data value among the measured values S230; recalculating of body temperature and oxygen saturation in the normal state if the error range of the body temperature and oxygen saturation is out of ±0.5° C. and ±1%, respectively, and calculating a deviation value between the body temperature value and oxygen saturation value in the normal state, and the measured values of body temperature and oxygen saturation collected thereafter S240; and classifying an infection stage according to a calculated deviation value range S250. The infection stage may be subdivided into a mild case and a severe case according to clinical diagnostic criteria.

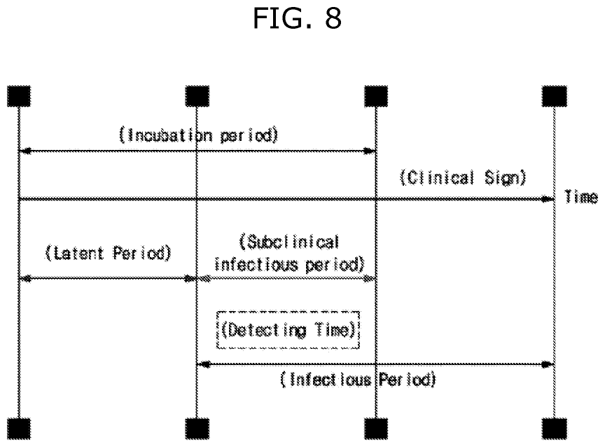

FIG. 8 is a configuration diagram illustrating reading of an infection time point of a target individual suspected of viral infection. Referring to FIG. 8, the target individual suspected of viral infection does not suffer a fever symptom in a latent period during an incubation period. According to the reading method of the present invention, the viral infection time point is read by tracking a fever condition from an initial point of fever to an end point of the incubation period after passing the latent period and the minimum quantitative threshold at which viremia appears. In particular, FIG. 8 shows reading of the infection time point in the asymptomatic infection period in which a fever condition is not recognized after infection with the novel virus COVID-19.

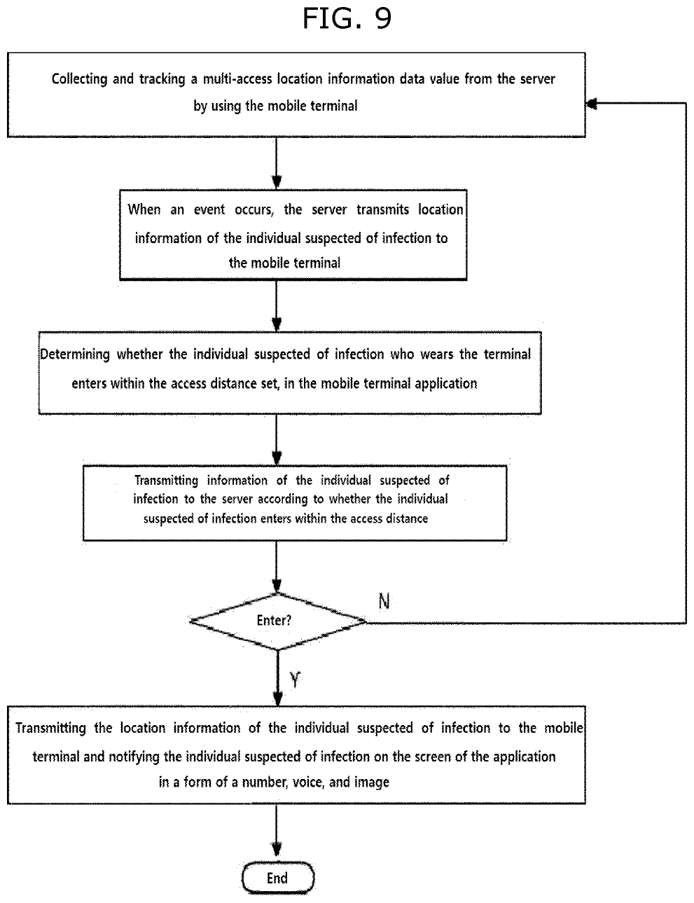

FIG. 9 is a flowchart illustrating a data flow state between the terminal 100 and the server 400 according to the present invention. Referring to FIG. 9, a multi-access location information data value is collected and tracked by the server 400 by using the mobile terminal 300a; when an event occurs, the server 400 transmits location information of the individual suspected of infection to the mobile terminal 300a; the mobile terminal application determines whether an individual suspected of infection who wears the terminal 100 enters within the access distance set in the mobile terminal application and transmits information of the individual suspected of infection to the server 400 according to whether the individual suspected of infection enters within the access distance; when the individual suspected of infection enters within the access distance, the server 400 transmits the location information of the individual suspected of infection to the mobile terminal 300a; the individual suspected of infection is notified on the screen of the application in a form of a number, voice, image, and others; and when an individual suspected of infection does not enter within the set access distance, the server 400 may continuously collect and track multiple access location information data values by using the mobile terminal 300a.

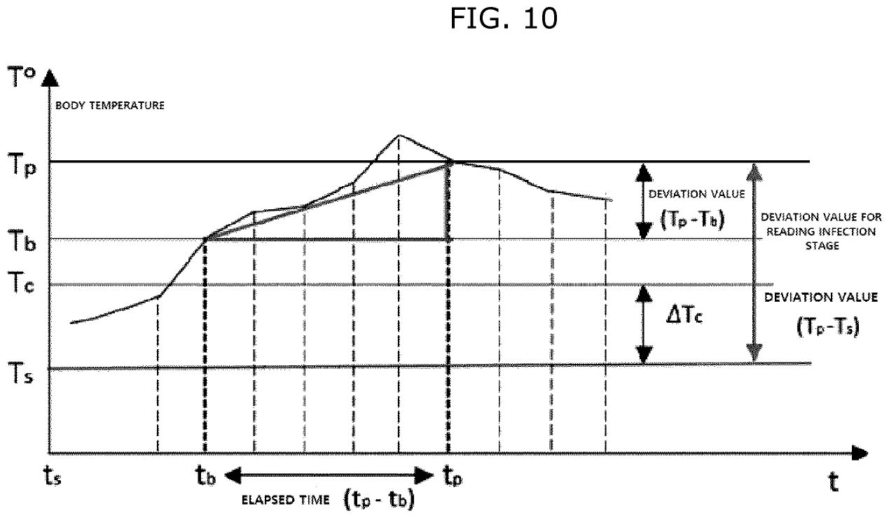

FIG. 10 is a graph schematically illustrating a method of calculating a deviation value and an increase and decrease rate of body temperature. Referring to FIG. 10, in order to determine whether the body temperature continues to rise before calculating the deviation value and the increase and decrease rate of the body temperature, the increase and decrease rate, which is a change in body temperature or a deviation value, over time is calculated and tracked, and in order to prevent distortion due to temporary increase and decrease in body temperature, it is determined whether the measured body temperature is normal by using the increase and decrease rate. The calculation formula is the deviation value divided by the elapsed time, and a change in body temperature per hour can be calculated. In the above calculation formula, Ts is defined as body temperature in a normal state (s: standard), Tp as current measured body temperature (p: present), Te as expected body temperature (e: expectation), to as an expected body temperature time, Δte as an expected body temperature reached value (e: expectation), Tc as body temperature change tracking standard temperature (c: criteria), ΔTc as an increase value of body temperature change tracking standard temperature (c: criteria), Tb as body temperature change tracking start temperature (b: beginning point), tb as a body temperature change tracking start time for (b: beginning point), tf as an expected fever onset time, Δtf as a fever onset reached value (f: fever), and ±Tmax as the maximum and maximum values of the increase and decrease rate. Calculation formulas of a deviation value and an increase and decrease rate from a body temperature change tracking start value (Tb) to a current measured body temperature value (Tp) are as following: an elapsed time (tp, tb) is (tp−tb), a deviation value (Tp, Tb) is (Tp−Tb), and an increase and decrease rate (Tp, Tb) is the deviation value (Tp, Tb) divided by the elapsed time, that is, (tp, tb)=(Tp−Tb)/(tp−tb). In addition, by using a deviation value between the body temperature value in the normal state and a measured body temperature value collected thereafter, infection stages are classified into an infection caution level from 0.0 to +1.0, an infection alert level from +1.0 to +2.0, and a suspicious infection level of +2.0 or higher, and each of the classified infection stages are further subdivided into from 0.0 to +0.5 as a mild level and from +0.5 to +1.0 as a severe level, from +1.0 to +1.5 as a mild level and from +1.5 to +2.0 as a severe level, and from +2.0 to +2.5 to +2.5 or higher as a severe level, respectively.

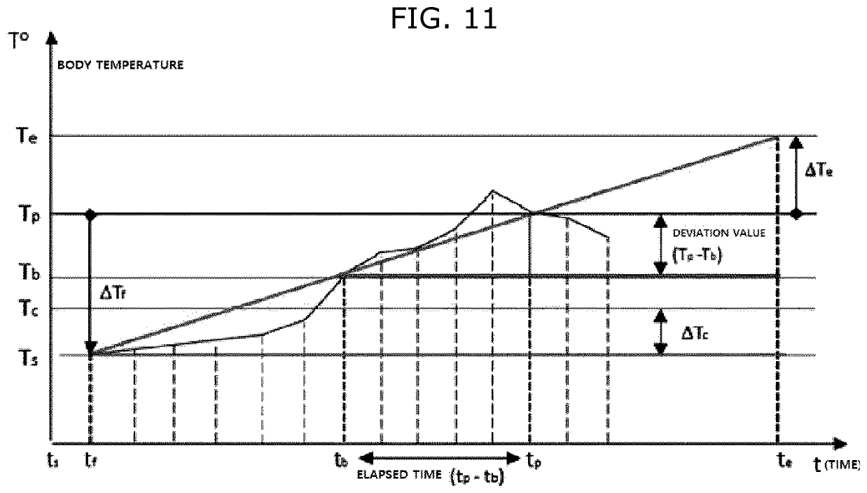

FIG. 11 is a graph schematically illustrating a method of tracking the expected time to reach body temperature in the infection stage and the expected time to the onset of fever. Referring to FIG. 11, a method of tracking the expected time to reach body temperature in the infection stage and the expected time to the onset of fever is as follows. In order to recognize the infection stage in advance, when body temperature rises above a certain level, tracking of the body temperature change starts and check a feverish condition in which the body temperature rises continuously for a certain period of time within the incubation period, a body temperature deviation value (increase and decrease value) that can be referenced when determining the level of infection stage, and an increase rate of body temperature (increase and decrease rate) over time. The deviation value of body temperature is a difference value between the body temperature value at the time of measurement and the body temperature value measured after a certain time has elapsed and represents a change in body temperature. The increase and decrease rate of body temperature is the body temperature deviation value (increase and decrease value) divided by the elapsed time and represents a change in body temperature per elapsed time. By using the calculated increase and decrease rate to indicate the increase rate of body temperature, the estimated time to reach body temperature at each infection stage and the estimated time to the actual fever onset can be tracked. The calculation formula for the estimated time to reach body temperature at each infection stage is as follows. The estimated time to reach the body temperature at the infection stage (te)=tp+the expected body temperature reached value (Ate), and Δte=(Te−Tp)/the increase and decrease rate (Tp, Tb). The calculation formula for the estimated time to the actual fever onset is as follows. The expected fever onset time (tf)=tp− the fever onset reached value (Δtf), and Δtf=(Tp−Ts)/the Increase and decrease rate (Tp, Tb).

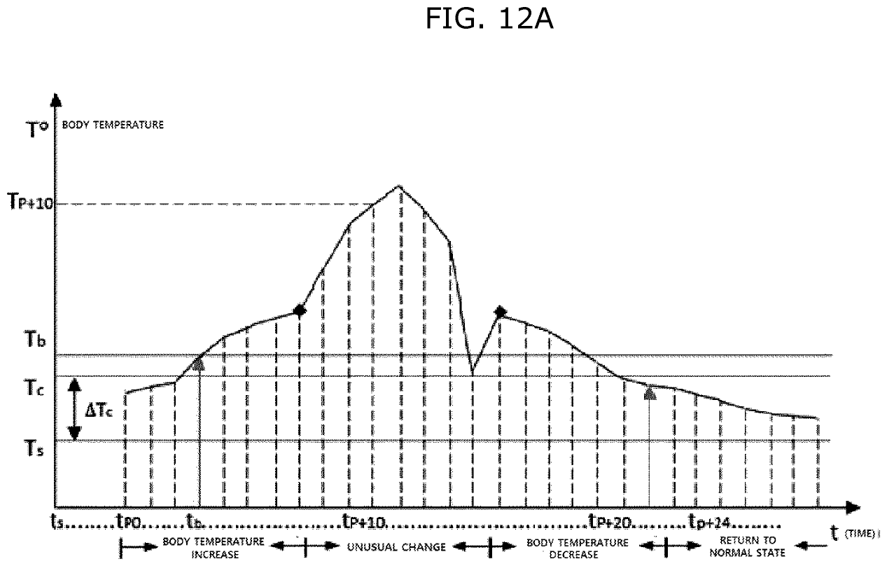

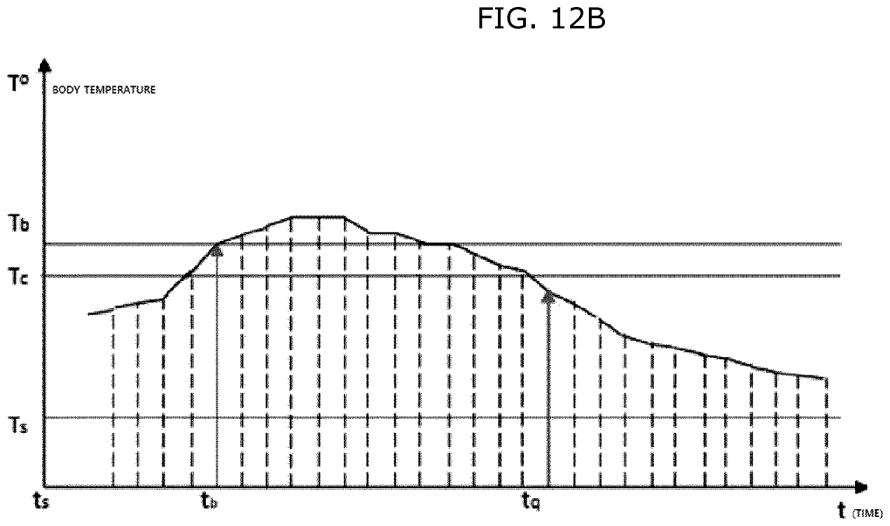

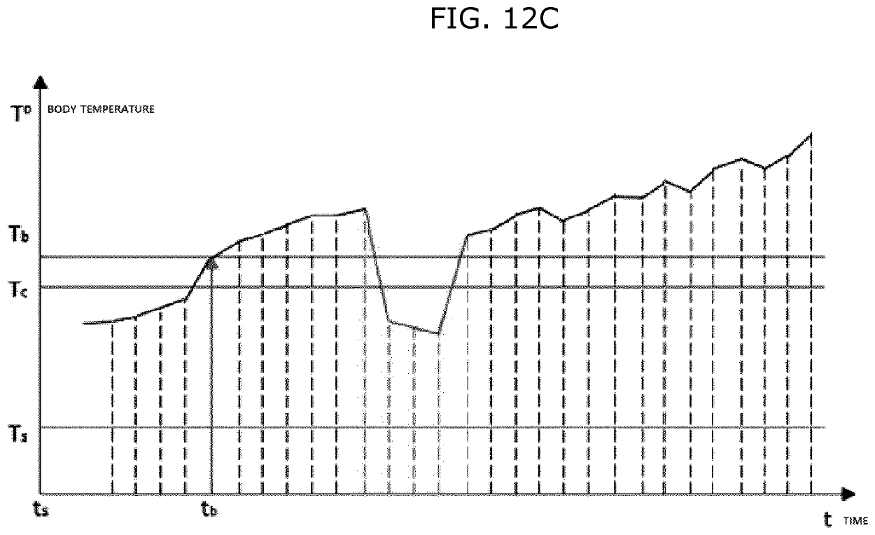

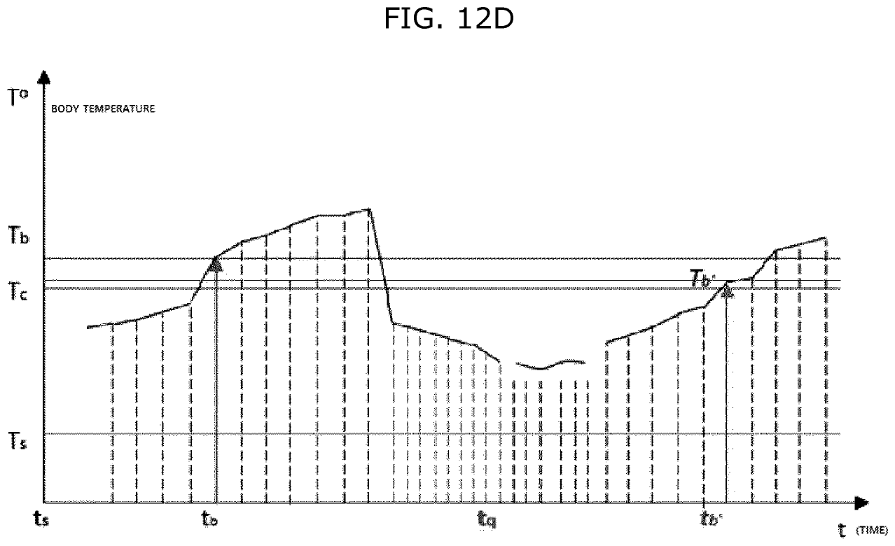

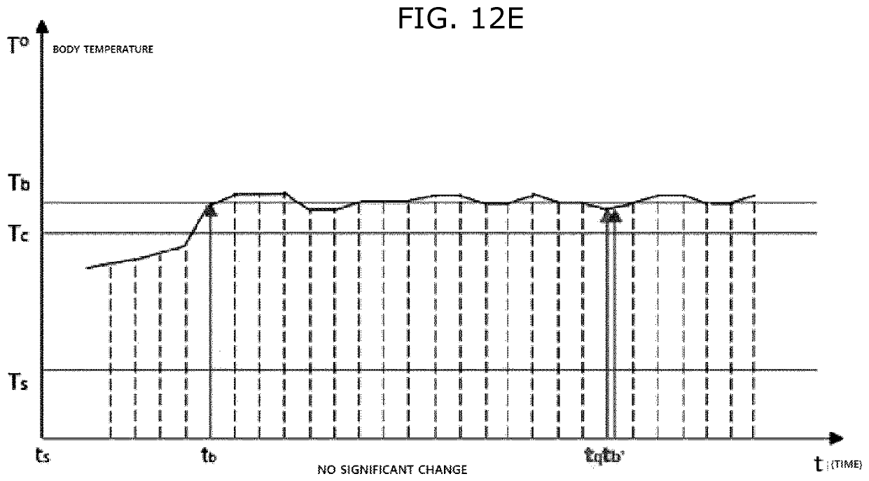

FIG. 12a is a graph schematically illustrating a method for tracking a change in body temperature. Referring to FIG. 12a, after checking the body temperature value (Ts) in the normal state, body temperature is automatically measured at the set time, such as in 1 hour time intervals. If the measured body temperature is higher than the body temperature change tracking reference body temperature (Tc=Ts+ΔTc), the tracking of body temperature change starts. If the measured body temperature is higher than the body temperature change tracking reference body temperature (Tc=Ts+ΔTc), the tracking of body temperature changes starts. When body temperature change tracking starts, the tracking is performed for a certain period of time to actually confirm an increase or decrease in body temperature and a return of body temperature to the normal state. Accordingly, it is possible to prevent an erroneous determination of viral infection due to an unusual increase or decrease in body temperature caused by a specific surrounding environment or non-daily activities. In this case, it is determined whether the change in body temperature has a characteristic of a continuous increase in body temperature of viral infection and whether the body temperature rises by 0.1 unit of a threshold value of a minimum measurement unit of a body temperature sensor.

FIGS. 12b, 12c, 12d, and 123 are graphs schematically illustrating a method of stopping tracking of a change in body temperature. Referring to FIG. 12b, a long lapse of time may cause distortion of the increase and decrease rate. In order to compensate for this, in a state where there is no significant body temperature change after the body temperature change tracking activity, if body temperature is lower than the body temperature change tracking reference body temperature (Tc), the tracking of body temperature change stops. Referring to FIG. 12c, in a state where there is a significant change in body temperature, if body temperature decreases below the body temperature change tracking reference temperature (Tc) and then increases above Tc, the tracking of body temperature change proceeds without interruption. Referring to FIG. 12d, if body temperature is continuously below Tc after a certain period of time (6h to 12h), the tracking of body temperature change stops. Referring to FIG. 12e, if body temperature maintains above the body temperature change tracking reference body temperature (Tc) after the body temperature change tracking starts, but there is no significant change for a certain period of time, that is, if the increase and decrease rate continues to decrease, this is recognized as an unusual state, the tracking of body temperature change stops and re-starts tracking a body temperature change from that point.

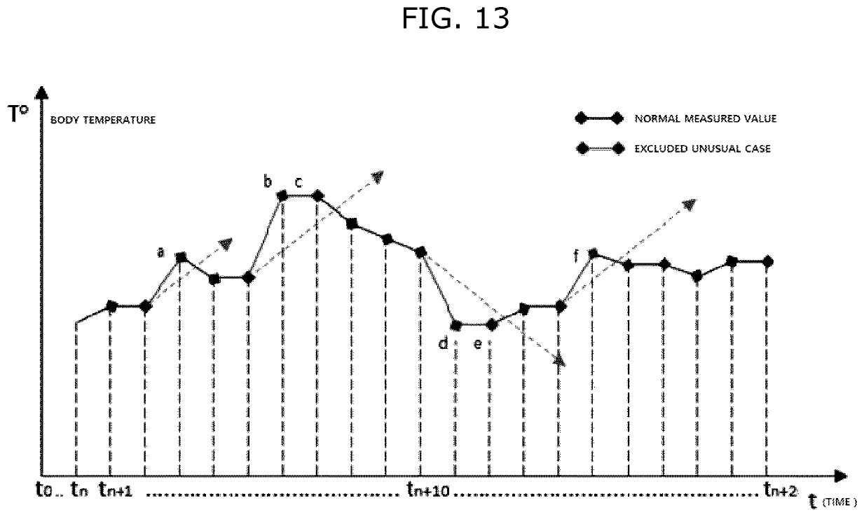

FIG. 13 is a graph for determining whether the measured body temperature is normal. Referring to FIG. 13, if an increase or decrease of a deviation value of the current measured body temperature value compared to the previous normal measured body temperature value is out of a range of ±Tmax, this is determined as an unusual case (a, b, c, and f of FIG. 13) and excluded. Alternatively, if a value obtained by applying ±Tmax body temperature increase and decrease rate to the previous normal measured body temperature is out of the range of values calculated for each elapsed time, this is determined as an unusual case (d and e of FIG. 13) and excluded.

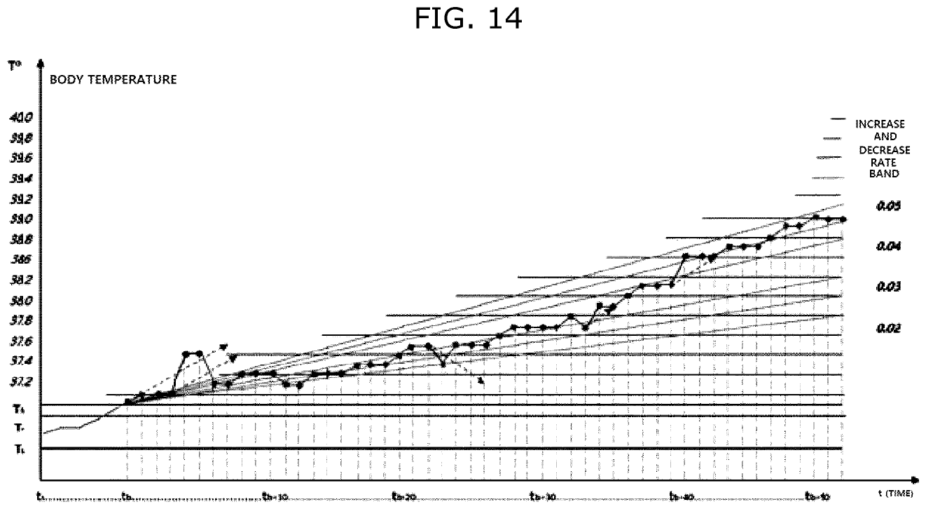

FIG. 14 is a graph schematically illustrating a method of predicting a virus type. Referring to FIG. 14, a method of predicting a virus type is as follows. Based on the body temperature values recognized as normal measured body temperature excluding the specific body temperature that are accumulated over time, virus types are classified by obtaining an average increase and decrease rate from the individually calculated increase and decrease rate. The average value of the individually calculated increase and decrease data value is calculated in 0.001 unit of the deviation value, and the virus type in a unit section of the increase and decrease band is predicted with a value obtained by rounding the average value to 0.01 unit. Therefore, in relation to a global pandemic, it is possible to perform epidemiological investigations such as the location of outbreak, identification of the source of infection, and the spread rate of viral infection according to the type of virus. In addition, by using the individually calculated increase and decrease rate of virus in an artificial intelligence method, it is possible to build a global digital anti-epidemic system based on a big data platform through machine learning and deep learning.

In addition, the present invention may further comprise the step of preventing viral infection. The step of preventing viral infection is performed by: downloading the mobile terminal application linked with the measurement terminal first; setting an access distance between the mobile terminal and a target individual suspected of viral infection who wears the biosignal measurement terminal to prevent viral infection; transmitting measured values such as body temperature (BT), oxygen saturation (SpO2), heart rate (HRM), and cough sound transmitted by using the Bluetooth module of the measurement terminal, to the mobile terminal; reading whether the event has occurred through clinical classification and combination of the measured values in the mobile terminal application; transmitting the individual information data value to the server when the event of an individual suspected of infection who wears the biosignal measurement terminal occurs; and notifying, by the server, the location information of the target individual suspected of infection who wears the biosignal measurement terminal and the number of the target individual of the event on a screen of the mobile terminal application by a form of text, voice, and image.

Simple modifications and variations of the present invention fall within the scope of the present invention, and the specific scope of protection of the present invention will be clarified by the appended claims.

What is claimed is:

1. A biosignal analysis method using a biosignal analysis system that establishes big data through machine learning and deep learning of a measured body temperature value generated by an event and that comprises:
a mobile terminal application analyzing a measured body temperature value measured from a biosignal measurement terminal and transmitted from a Bluetooth module of the biosignal measurement terminal to determine whether the event has occurred;
a server receiving the measured body temperature value analyzed in the mobile terminal application when the event occurs; and
at least one of a mobile terminal receiving and wirelessly transmitting location data and a measured body temperature value wirelessly and a gateway receiving and transmitting the location data and the measured body temperature value,
wherein the server receives and stores the location data and the measured body temperature value from at least one of the mobile terminal and the gateway; receives the location data and the measured body temperature value when the event occurs; includes multiple access location information of the mobile terminal application; and notifies a number, text, image, and voice of individual information of the event,
wherein the server further includes: a database unit analyzing and storing the measured body temperature value and a transceiver unit capable of transmitting the measured body temperature value through an internet network,
wherein the biosignal analysis method comprises the steps of:
calculating a body temperature value in a normal state by using an analysis algorithm of a body temperature value measured from the bio-signal measurement terminal;
determining whether the measured body temperature value is normal;
classifying an infection stage by using a deviation value between the calculated body temperature value in the normal state and a subsequently measured body temperature value;
calculating a deviation value and an increase and decrease rate of the measured body temperature value;
tracking an expected time of reaching to body temperature in an infection stage and an expected time of an onset of fever in the infection stage;
tracking a change in body temperature by using an increase and decrease rate of the deviation value of body temperature;
stopping the tracking of the change in body temperature; and
predicting a virus type through an increase and decrease of the measured body temperature value,
wherein the step of calculating the deviation value and the increase and decrease rate body temperature includes steps of: calculating and tracking the increase and decrease rate, which is a change in body temperature or a deviation value of body temperature, over time in order to determine whether body temperature continuously increases; and determining whether the measured body temperature value is normal by using the increase and decrease rate in order to prevent distortion due to a temporary increase and decrease in body temperature,
wherein in the step of calculating the deviation value, a change in body temperature per hour is calculated by a calculation formula dividing a deviation value by an elapsed time, wherein the deviation value and the increase and decrease rate from a body temperature change tracking start value (Tb) to a current measured body temperature value (Tp) are calculated by calculation formulas in which an elapsed time (tp, tb) is (tp−tb), the deviation value (Tp, Tb) is (Tp−Tb), and the increase and decrease rate (Tp, Tb) is the deviation value (Tp, Tb) divided by the elapsed time (tp, tb), that is, (tp, tb)=(Tp−Tb)/(tp−tb).

2. The biosignal analysis method of claim 1, wherein the step of calculating the body temperature value in the normal state includes the steps of:
performing temperature acclimatization between a body temperature sensing element and a skin surface in a static state for a certain period of time;
measuring body temperature several times at regular time intervals within an error range of ±0.5° C.; and
calculating an average value of the measured body temperature values excluding the highest measured body temperature and lowest measured body temperature, as a body temperature value in the normal state.

3. The biosignal analysis method of claim 1, wherein the step of determining whether the measured body temperature value is normal is performed by:
determining and excluding an unusual case in which an increase and decrease value of a deviation value of the current measured body temperature value compared to a previous normal measured body temperature value is out of ±Tmax range; or
determining and excluding an unusual case in which a value obtained by applying ±Tmax range of body temperature increase and decrease rate to a previous normal measured body temperature is out of a range of value calculated for each elapsed time.

4. The biosignal analysis method of claim 1, wherein the step of classifying the infection stage uses the deviation value between the body temperature value in the normal state and a measured body temperature value collected thereafter and is performed by:
dividing infection stages into an infection caution level from 0.0 to +1.0, an infection alert level from +1.0 to +2.0, and a suspicious infection level of +2.0 or higher; and
subdividing each of the divided infection stages into from 0.0 to +0.5 as a mild level and from +0.5 to +1.0 as a severe level, from +1.0 to +1.5 as a mild level and from +1.5 to +2.0 as a severe level, and from +2.0 to +2.5 to +2.5 or higher as a severe level, respectively.

5. The biosignal analysis method of claim 1, wherein in order to recognize the infection stage in advance, the step of tracking the expected time of reaching to body temperature in the infection stage and the expected time of the onset of fever in the infection stage is performed by tracking the change in body temperature when body temperature rises above a certain level, in order to check a fever condition in which body temperature rises continuously for a certain period of time within an incubation period, the body temperature deviation value, which is a body temperature increase and decrease value, and an increase rate of body temperature, which is a body temperature increase and decrease rate, over time,
wherein the deviation value is a difference between a body temperature value at a time of measurement and a body temperature value measured after a certain period of time has elapsed and represents the change in body temperature,
wherein the increase and decrease rate is a change in body temperature per elapsed time, is calculated by dividing the deviation value, which is the increase and decrease value, by the elapsed time, and represents an increase rate in body temperature,
wherein the estimated time of reaching body temperature at each of infection stages and the estimated time of the onset of fever are tracked by utilizing the calculated increase and decrease rate by using a calculation formular in which the estimated time (te) of reaching body temperature at infection stage is [tp+(Te−Tp)] divided by the increase and decrease rate (Tp, Tb) and by using a calculation formular in which the estimated time (tf) of the onset of fever is [tp−(Tp−Ts)] divided by the increase and decrease rate (Tp, Tb).

6. The biosignal analysis method of claim 1, wherein the step of tracking the change in body temperature is performed by:
checking the body temperature value (Ts) in the normal state first;
then automatically measuring body temperature at a set time;
then starting tracking of a change in body temperature if the measured body temperature is higher than body temperature change tracking reference body temperature (Tc=Ts+ΔTc);
proceeding the tracking for a certain period of time to confirm an actual increase and decrease of body temperature and a return of body temperature to the normal state once the tracking of the change in body temperature starts and to prevents erroneous determination caused by unusual temperature fluctuations due to unusual surroundings or non-daily activities; and
determining whether the change in body temperature has a characteristic of a continuous increase in body temperature of viral infection and whether the body temperature rises by 0.1 unit of a threshold value of a minimum measurement unit of a body temperature sensor.

7. The biosignal analysis method of claim 1, wherein in order to compensate for distortion of the increase and decrease rate over a long period of time, the step of stopping the tracking of the change in body temperature is performed by:
in a state that there is no significant change in body temperature after the tracking of the change in body temperature, if the measured body temperature is below a tracking reference temperature (Tc) of the change in body temperature; or stopping the tracking, stopping the tracking;
in a state that there is a significant change in body temperature, if the measured body temperature is below the body temperature change tracking reference body temperature (Tc) of the change in body temperature and is kept to be below the tracking reference body temperature (Tc) after a certain period of time, while not stopping the tracking if the measured body temperature increases above the tracking reference body temperature (Tc) after a certain period of time after the measured body temperature is below the tracking reference body temperature (Tc), stopping the tracking;
in a state in which the measured body temperature is kept being higher than the tracking reference temperature (Tc) but there is no significant change for a certain period of time, that is, in a state in which the rate of increase and decrease continuously decreases after starting the tracking of the change in body temperature, recognizing the state as an unusual state, stopping the tracking, and re-tracking the change in body temperature from a corresponding point.

8. The biosignal analysis method of claim 1, wherein predicting the virus type is performed by:
classifying the virus type by obtaining an average increase and decrease rate from the increase and decrease rate individually calculated with body temperature value excluding unusual body temperature, accumulated over time, and recognized as normal body temperature;
calculating an average value of the individually calculated increase and decrease data values in 0.001 unit of a deviation value; and
predicting a unit section of an increase and decrease rate band with a value obtained by rounding the average value to 0.01 unit.

9. The biosignal analysis method of claim 1, further comprising the step of reading an infection time of a target individual suspected of viral infection,
wherein the step of reading an infection time is performed by:
calculating an average body temperature value in the normal state;
calculating a deviation value between the average body temperature value and a measured body temperature value collected from the onset of fever to an end of an incubation period, wherein the onset of fever is a point from which body temperature starts to increase due to an immune response after a minimum quantitative threshold of the virus in the blood as the virus passes through a latent period and spreads after viral infection; and
reading the infection time when the deviation value greater than a significance level is derived.

10. The biosignal analysis method of claim 1, further comprising the step of preventing viral infection,
wherein the step of preventing viral infection is performed by:
downloading the mobile terminal application linked with the biosignal measurement terminal first;
setting an access distance between the mobile terminal and a target individual suspected of viral infection who wears the biosignal measurement terminal to prevent viral infection;
transmitting measured values such as body temperature (BT), oxygen saturation (SpO2), heart rate (HRM), and cough sound transmitted by using the Bluetooth module of the biosignal measurement terminal, to the mobile terminal;
reading whether the event has occurred through clinical classification and combination of the measured values in the mobile terminal application;
transmitting a data value of the individual information to the server when the event of an individual suspected of infection who wears the biosignal measurement terminal occurs; and
notifying, by the server, the location information of the target individual suspected of infection who wears the biosignal measurement terminal and the number of the target individual of the event on a screen of the mobile terminal application by a form of text, voice, and image.

11. The biosignal analysis method of claim 1, further comprising the steps:
collecting biosignal measurement data values in a static state after temperature acclimatization between a body temperature sensing element and a skin surface for a certain period of time after wearing the biosignal measurement terminal, in order to prevent an error in a data value and distortion of a measured data value according to a shape of the biosignal measurement terminal, a user condition, and a surrounding environment; and
checking a wearing state of the biosignal measurement terminal through the mobile terminal application of an individual who wears the biosignal measurement terminal, if a measured data value cannot be received by the mobile terminal due to movement of the individual wearing the terminal,
wherein a lower surface of the biosignal measurement terminal includes a groove of a certain shape into which an open type chamber is mount, and a bottom surface of the open type chamber includes an embossed and engraved shape to prevent sliding with the skin surface and to block an external light.
